(19) United States
(12) Patent Application Publication
(10) Pub. No.: US 2026/0276531 A1
(43) Pub. Date: Sep. 17, 2026

(54) METHODS AND SYSTEMS FOR PREDICTING ABSORBANCE OF A WATER SAMPLE USING A MACHINE LEARNING MODEL

(71) Applicants: John Benedict Allasas Estrada, Fresno, CA (US); Pauline Victoria Allasas Estrada, Fresno, CA (US); Achuth Vinay, Fresno, CA (US); Arnav Gupta, Fresno, CA (US)

(72) Inventors: John Benedict Allasas Estrada, Fresno, CA (US); Pauline Victoria Allasas Estrada, Fresno, CA (US); Achuth Vinay, Fresno, CA (US); Arnav Gupta, Fresno, CA (US)

(21) Appl. No.: 19/568,720

(22) Filed: Mar. 16, 2026

Related U.S. Application Data
(60) Provisional application No. 63/772,348, filed on Mar. 14, 2025.

(51) Int. Cl. G01N 21/31; G01N 33/18; G06T 7/00; H04N 23/56

(52) U.S. Cl. CPC G01N 21/31; G01N 33/1826; G06T 7/0002; H04N 23/56; G06T 2207/20081; G06T 2207/20084

(57) ABSTRACT

A method of analyzing a water sample includes capturing a spectrally dispersed image of light transmitted through the water sample, generating image data corresponding to the image, providing the data to a trained machine-learning regression model, and outputting from the model a predicted absorbance value of the water sample at a selected wavelength. A machine-learning regression model can be trained using a plurality of pairs of data corresponding to a training image and a reference absorbance measurement from a reference spectrophotometer. A system for analyzing a water sample can include a light source, an imaging spectrometer having an image sensor and an optical dispersive element, and at least one processor having instructions to cause the processor to execute a machine learning regression model.

### CROSS-REFERENCE TO RELATED APPLICATIONS

This application claims priority to, and the benefit of, U.S. Provisional Patent Application No. 63/772,348, filed Mar. 14, 2025, which is hereby incorporated by reference in its entirety.

### FIELD OF THE INVENTION

The present invention concerns training and use of machine learning models useful for environmental monitoring of water bodies. More particularly, embodiments of the present invention concern a machine-learning regression model receiving image data corresponding to a spectrally dispersed image of light transmitted through a water sample as an input and outputting a predicted absorbance value.

### BACKGROUND OF THE INVENTION

Algae are natural components of marine and freshwater flora and perform many roles that are important to healthy ecosystems. However, when conditions are favorable, algae can rapidly multiply into excessive quantities commonly referred to as “blooms.” When blooms, or dense surface scum, are formed, the risk of toxin contamination of surface water can increase, especially when cyanobacteria are present. Cyanobacteria are photosynthetic microscopic bacteria that are of particular concern because of their potential impacts on drinking and recreational waters. Harmful algal blooms (HABs) can therefore pose serious environmental, economic, and public-health risks.

The monitoring and detection of algae and cyanobacteria in surface water, wastewater, and other water samples is important for water-quality management and for the early identification of bloom conditions. One common conventional approach for monitoring potential algal blooms is remote sensing, such as satellite-based imaging. Remote sensing can provide broad spatial coverage over large areas, but it may require expensive equipment, complex calculations, and may be affected by atmospheric and other environmental conditions. In contrast, in situ techniques can provide more localized, higher-resolution data and can be better suited for real-time or near-real-time monitoring of particular water samples.

A variety of in situ methods have been used to assess algal and cyanobacterial content in water samples. These include direct cell counting, spectrophotometry, dry-weight measurements, and chlorophyll extraction techniques. Spectrophotometric measurements, including absorbance-based techniques, are among the more common and reliable methods for quantifying algal or cyanobacterial characteristics in water. In many instances, absorbance at a wavelength associated with chlorophyll absorption may provide a useful indicator of algal or cyanobacterial presence in a sample. However, conventional spectrophotometric approaches may require laboratory instruments, equipment, and materials that are not always readily available or convenient for routine field use. It is also important to note multiple cyanobacteria species use not only visible light but can access far-red, near-infrared and UV light spectrum for photosynthesis by switching from their normal form of chlorophyll (known as chlorophyll a) to an alternate form (chlorophyll f), which have different absorbance wavelengths. In doing so, it allows the species to survive in changing conditions, but also complicates conventional assessments of water samples.

With the continued development of image-based analysis and machine-learning techniques, there remains a need for systems and methods that can rapidly analyze a water sample using spectral image data and output a predicted absorbance value at a selected wavelength without requiring a reference spectrophotometer during runtime operation. There further exists the need for lower-cost and more accessible tools that can provide localized analysis of water samples and that may be used, in some embodiments, to estimate algal or cyanobacterial concentration, including cyanobacteria concentration, and/or to assist in monitoring conditions associated with harmful algal blooms.

### BRIEF SUMMARY OF THE INVENTION

In accordance with some embodiments of the present invention, a method of analyzing a water sample may comprise: a) capturing, with an imaging spectrometer which may comprise an image sensor and an optical dispersive element, a spectrally dispersed image of light transmitted through the water sample, wherein the optical dispersive element may be arranged such that the light transmitted through the water sample forms the spectrally dispersed image on the image sensor; b) generating image data corresponding to the spectrally dispersed image; c) providing at least a portion of the image data to a trained machine-learning regression model; and d) outputting, with the trained machine-learning regression model, a predicted absorbance value of the water sample at a selected wavelength. In some embodiments, the step of capturing the spectrally dispersed image may comprise illuminating the water sample with a full-spectrum light-emitting diode. In some embodiments, the optical dispersive element may comprise a diffraction spectroscope. In some embodiments, the image sensor may be adapted to detect light having wavelengths between about 300 and about 1,100 nm. In some embodiments, the image data may include wavelength content in a range from about 300 nm to about 1,100 nm. In some embodiments, the selected wavelength may be about 680 nm. In some embodiments, the selected wavelength may correspond to chlorophyll absorption. In some embodiments, the method of analyzing the water sample may further comprise using the predicted absorbance value to predict cyanobacteria concentration in the water sample.

In accordance with some embodiments of the present invention, a method of training a machine-learning model to predict absorbance of a water sample at a selected wavelength may comprise: a) for each of a plurality of water samples, 1) capturing, from an imaging spectrometer which may comprise an image sensor and an optical dispersive element, a spectrally dispersed image of light transmitted through the water sample; 2) obtaining, from a reference spectrophotometer, a reference absorbance measurement of the water sample at the selected wavelength; 3) storing a paired training record which may comprise data corresponding to the spectrally dispersed image and the reference absorbance measurement; and b) training, using the paired training records, a machine-learning regression model which may be configured to output a predicted absorbance value at the selected wavelength. In some embodiments, the selected wavelength may be about 680 nm. In some embodiments, the paired training records may be acquired every two to three days over about two weeks. In some embodiments, each water sample may be derived from one of at least two independent sources. In some embodiments, each water sample may comprise a dilution of one of about 33%, about 66%, and about 100% of the source. In some embodiments, about four of the plurality of water samples may be derived from the same source and may comprise the same dilution. In some embodiments, the machine-learning regression model may comprise a convolutional neural network regression model which may be configured as a sequential model. In some embodiments, the convolutional neural network regression model may comprise three convolutional 2D layers, two dense layers, and a dropout layer. In some embodiments, training the machine-learning regression model may comprise using Adam as an optimizer, ReLU as an activation function, mean squared error as a loss function, and early stopping to prevent overfitting. In some embodiments, about eighty percent (80%) of the paired training records may be used as a training data set and about twenty percent (20%) of the paired training records may be used as a validation data set.

In accordance with some embodiments of the present invention, a system for analyzing a water sample may comprise: a) an illumination source which may be arranged to direct light through the water sample; b) an imaging spectrometer which may comprise an image sensor and an optical dispersive element arranged such that the light transmitted through the water sample forms a spectrally dispersed image on the image sensor; and c) at least one processor operatively engaged with a memory storing instructions that, when executed by the at least one processor, cause the at least one processor to: 1) receive image data corresponding to the spectrally dispersed image, 2) execute a trained machine-learning regression model using at least a portion of the image data as input, and 3) output a predicted absorbance value for the water sample at a selected wavelength. In some embodiments, the illumination source may comprise a full-spectrum light-emitting diode, the optical dispersive element may comprise a diffraction spectroscope, the image sensor may be adapted to detect light having wavelengths between about 300 and about 1,100 nm, and the selected wavelength may be about 680 nm.

### BRIEF DESCRIPTION OF THE FIGURES

FIG. 1 is a diagram illustrating an exemplary system for analyzing a water sample, in accordance with some embodiments of the present invention.

FIG. 2 is a flowchart illustrating an exemplary process for training and validating a neural network model, in accordance with some embodiments of the present invention.

FIG. 3 is an image illustrating an exemplary spectrally dispersed image, in accordance with some embodiments of the present invention.

### DETAILED DESCRIPTION OF THE INVENTION

The invention, in its various aspects, will be explained in greater detail below. While the invention will be described in conjunction with several exemplary embodiments, the exemplary embodiments themselves do not limit the scope of the invention. Similarly, the exemplary illustrations in the accompanying drawings, where like elements have like numerals, do not limit the scope of the exemplary embodiments and/or invention, including any length, angles, or other measurements provided. Rather the invention, as defined by the claims, may cover alternatives, modifications, and/or equivalents of the exemplary embodiments.

### Overview

In some aspects, the present invention provides systems, apparatuses, and methods for analyzing a water sample by capturing a spectrally dispersed image of light transmitted through the water sample and using a machine-learning regression model to output a predicted absorbance value of the water sample at a selected wavelength. In some embodiments, the selected wavelength may correspond to chlorophyll absorption and may be about 680 nm. Although the model output in preferred embodiments is a predicted absorbance value at a selected wavelength, it is to be appreciated that the predicted absorbance value may then be used, alone or in combination with additional information, to estimate algal or cyanobacteria concentration and/or assess conditions associated with a harmful algal bloom.

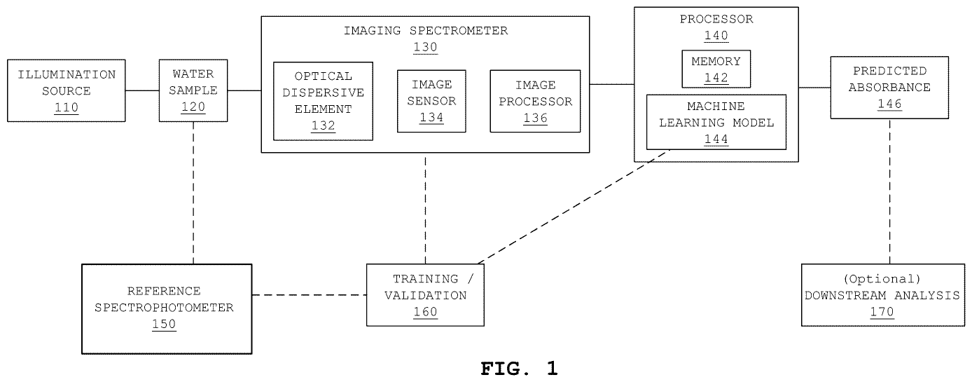

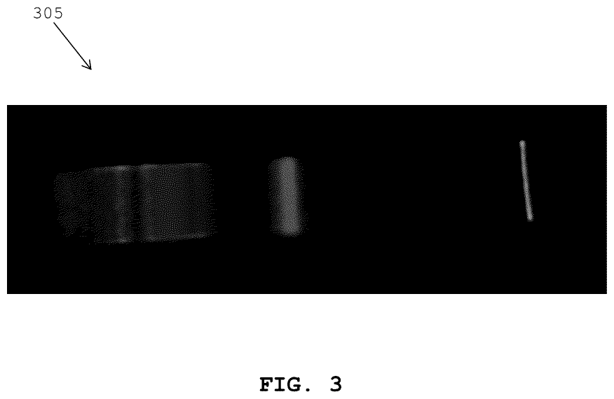

For example, and with reference to FIG. 1, an exemplary system 100 may include an illumination source 110 arranged to direct light through a water sample 120. In some implementations, illumination source 110 may comprise a light-emitting diode and may be dimmable. In preferred implementations, illumination source 110 is a full-spectrum light. It is to be appreciated that illumination source, preferably, emits wavelengths corresponding to the target algae or cyanobacteria photosynthesis wavelengths. Light transmitted through water sample 120 may be received by an imaging spectrometer 130. Imaging spectrometer 130 may comprise an optical dispersive element 132 and an image sensor 134 arranged such that the light transmitted through water sample 120 forms a spectrally dispersed image on image sensor 134. Although the spectrally dispersed image may depend on the particular construction of imaging spectrometer 130, FIG. 3 illustrates one example of spectrally dispersed image 305. Although FIG. 1 illustrates one exemplary arrangement, the relative positions of illumination source 110, water sample 120, optical dispersive element 132, and image sensor 134 may vary provided that transmitted light from water sample 120 is spectrally separated and imaged at image sensor 134.

In some embodiments, optical dispersive element 132 may comprise a diffraction spectroscope, diffraction grating, prism, or other optic configured to separate incident light by wavelength. In some embodiments, image sensor 134 may comprise a CCD or CMOS sensor and may be adapted to detect light having wavelengths between about 300 nm and about 1,100 nm. In some implementations, image sensor 134 may be sensitive to near infrared wavelengths by removal or omission of an infrared-cut filter. Imaging spectrometer, in some embodiments, may comprise image processor 136 operatively engaged with image sensor 134 for preparing image data therefrom. In some embodiments, the image data may be in the form of a digitally readable image file, for example and without limitation, a jpg, bmp, tiff, or other file type, that may be further processed, including but not limited to normalization, cropping, compression. In preferred embodiments, the image data generated from imaging spectrometer 130 may include wavelength content in a range from about 300 nm to about 1,100 nm.

Image data corresponding to the spectrally dispersed image may be provided to one or more processors 140 operatively engaged with memory 142. In some implementations, processor 140 may be disposed in or coupled to the imaging spectrometer, such as in an embedded computing or edge device, or similar computing platform. In some embodiments, processor 140 may communicate with a separate computing device, server, smartphone, tablet, laptop, or cloud resource over a wired or wireless network. Memory 142 may store program instructions and a trained machine-learning regression model 144. In some embodiments, the machine-learning regression model 144 may receive at least a portion of the image data as input and may output a predicted absorbance value 146 for water sample 120 at the selected wavelength. In preferred embodiments, machine-learning regression model 144 provides a quantitative absorbance prediction rather than a binary classification output.

Before runtime use, machine-learning regression model 144 may be trained using paired training records that associate spectral image information with reference absorbance measurements. As further illustrated in FIG. 1, an optional reference spectrophotometer 150 may be used during model development to obtain a reference absorbance measurement of a water sample at the selected wavelength. In some embodiments, reference spectrophotometer 150 is used for training and/or validation only and is not required when the trained model is later used to analyze a new sample. A training/validation module 160 may store paired training records including data corresponding to a spectrally dispersed image (for example, from imaging spectrometer 130) and a corresponding reference absorbance measurement (for example, from reference spectrophotometer 150), and may use those paired training records to train and validate machine-learning regression model 144.

In some embodiments, water samples used for training may be collected from a plurality of independent sources so that the training set spans different algae conditions, microbial communities, and absorbance levels. In some embodiments, water samples may be collected from at least two independent sources. In some embodiments, the water samples may be diluted to about 33%, about 66%, and about 100% of a source sample (100% meaning that the sample is not diluted). In some embodiments, about four water samples may be prepared from the same source at the same dilution. The samples may be maintained under controlled conditions and aliquots may be periodically analyzed over time. For example, and without limitation, paired training records may be acquired every two to three days over about two weeks so that the training data spans a progression from relatively low absorbance values to relatively high absorbance values. It is to be appreciated that different aliquots from the same preparation (source and dilution) may express different absorbance values depending on whether the sample contains algae or cyanobacteria that is replicating or “blooming”. For each aliquot, a spectrally dispersed image may be captured using imaging spectrometer 130 and a corresponding reference absorbance measurement at the selected wavelength may be obtained using reference spectrophotometer 150.

In some embodiments, at least a portion of the captured image data may be preprocessed by processor 140 before being supplied to machine-learning regression model 144. Such preprocessing may include cropping or isolating a region of interest corresponding to the dispersed spectral band, resizing, normalization, intensity correction, and/or wavelength calibration. In preferred embodiments, machine-learning regression model 144 may comprise a convolutional neural network regression model configured as a sequential model. In some implementations, the model may include three convolutional 2D layers, two dense layers, and a dropout layer. Training may include use of Adam as an optimizer, ReLU as an activation function, mean squared error as a loss function, and/or early stopping to prevent overfitting. In some embodiments, about eighty percent of the paired training records may be used as a training data set and about twenty percent may be used as a validation data set. It is to be appreciated that the foregoing model architecture and training parameters are exemplary and non-limiting, and other regression architectures capable of mapping image data to absorbance values may be employed in accordance with some embodiment of the present invention.

Once trained, machine-learning regression model 144 may be executed using image data from a new water sample that was not part of the training or validation data. In some embodiments, the trained model may be incorporated into a prediction output program stored in memory 142 that receives image data from imaging spectrometer 130 and outputs predicted absorbance value 146. It is to be appreciated that, although reference spectrophotometer 150 is illustrated in FIG. 1, it is not required for run-time operation. The predicted absorbance value may be displayed, stored, transmitted, or compared with one or more thresholds. In some embodiments, the selected wavelength may correspond to chlorophyll absorption and may be about 680 nm, such that the predicted absorbance value may serve as an indicator associated with algae or cyanobacteria in the water sample.

As also shown in FIG. 1, predicted absorbance value 146 may be provided to an optional downstream analysis module 170. In some embodiments, downstream analysis module 170 may use predicted absorbance value 146 to estimate cyanobacteria concentration in water sample 120 or may predict the likelihood of a harmful algal bloom. In some embodiments, predicted absorbance values generated from samples collected from the same water source over time may be analyzed to evaluate changes in water conditions and to identify a possible increase in bloom-related activity. Thus, while the trained machine-learning regression model 144 preferably outputs a predicted absorbance value at the selected wavelength, that output may be used as a proxy or input for determining the possible presence of cyanobacteria, estimating concentration, triggering additional testing, or supporting prediction, early warning, and/or monitoring of harmful algal bloom conditions.

### Methods of Analyzing a Water Sample

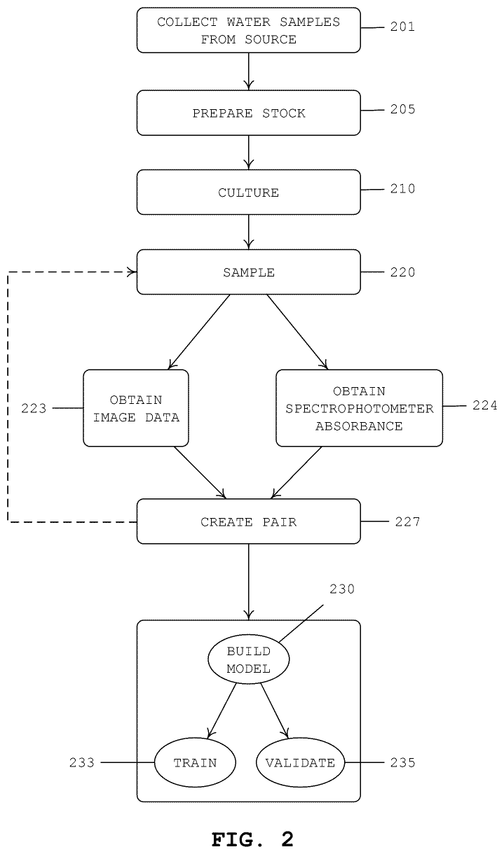

According to some embodiments of the present invention, and with reference to FIGS. 1 and 2, once trained machine-learning regression model 144 has been developed and validated, including but not limited to by method 200 of FIG. 2, system 100 of FIG. 1 may be used to analyze a water sample 120. More particularly, a method of analyzing water sample 120 may include capturing, with imaging spectrometer 130, a spectrally dispersed image of light transmitted through water sample 120. Optical dispersive element 132 may be arranged such that the light transmitted through water sample 120 forms the spectrally dispersed image on image sensor 134. In some embodiments, imaging spectrometer 130 may further comprise image processor 136 configured to convert output from image sensor 134 into image data corresponding to the spectrally dispersed image received by image sensor 134. In some embodiments, image processor 136 and/or processor 140 may further process such image data. At least a portion of the image data may be provided to trained machine-learning regression model 144 stored in memory 142 and executed by processor 140. In response, trained machine-learning regression model 144 may output predicted absorbance value 146 of water sample 120 at a selected wavelength. In some embodiments, the portion of image data provided to trained machine-learning regression model 144 may correspond to a cropped or otherwise processed region containing the dispersed spectral band.

In some embodiments, capturing the spectrally dispersed image may include illuminating water sample 120 with illumination source 110 arranged to direct light through water sample 120 toward imaging spectrometer 130. In preferred embodiments, illumination source 110 comprises a full-spectrum light-emitting diode and may provide backlighting of water sample 120 during image capture. In some implementations, illumination source 110 may be dimmable so that image acquisition conditions may be controlled. In some implementations, optical dispersive element 132 comprises a diffraction spectroscope. Image sensor 134 may be adapted to detect light having wavelengths between about 300 nm and about 1,100 nm. Similarly, the image data generated from the spectrally dispersed image may include wavelength content in a range from about 300 nm to about 1,100 nm.

In preferred embodiments, the selected wavelength corresponds to chlorophyll absorption wavelengths in target algae and/or cyanobacteria for analysis. In preferred implementations, the selected wavelength may be about 680 nm. Thus, in some implementations, trained machine-learning regression model 144 may be configured to output predicted absorbance value 146 representing a predicted absorbance of water sample 120 at about 680 nm based on the spectrally dispersed image captured by imaging spectrometer 130. It is to be appreciated that some embodiments of the present invention are not limited to any particular selected wavelengths, and other selected wavelengths associated with pigments, organisms, or analytes of interest may likewise be used in other embodiments. It is also to be appreciated that, in some embodiments, the model may be trained and adapted to predict absorbance at more than one wavelength for a given sample.

In some embodiments, and particularly where the selected wavelength corresponds to chlorophyll absorption, the method may further include using predicted absorbance value 146 to predict cyanobacteria concentration in water sample 120. For example, predicted absorbance value 146 may be evaluated by processor 140 or supplied to an optional downstream analysis 170 to estimate cyanobacteria concentration based on a relationship between absorbance at the selected wavelength and cyanobacteria content in the sample. In some implementations, repeated application of the method to additional water samples obtained from the same water body at different times may permit changes in predicted absorbance value 146 and/or predicted cyanobacteria concentration to be monitored over time, which may be useful for identifying developing bloom conditions, triggering additional testing, or informing harmful algal bloom monitoring.

### Methods for Training Machine-Learning Models

According to some embodiments of the present invention, and with reference to FIGS. 1 and 2, method 200 may be used to train a machine-learning regression model to predict absorbance of a water sample at a selected wavelength. More particularly, a method of training machine-learning regression model 144 may include, for each of a plurality of water samples, capturing a spectrally dispersed image (such as the exemplary spectrally dispersed image 305 of FIG. 3) from imaging spectrometer 130 and obtaining a reference absorbance measurement of the water sample at the selected wavelength from reference spectrophotometer 150, storing a paired training record comprising data corresponding to the spectrally dispersed image and the reference absorbance measurement, and training machine-learning regression model 144 using a plurality of paired training records to output a predicted absorbance value at the selected wavelength.

In some embodiments, and in accordance with Step 201 of FIG. 2, water may be collected from one or more sources to provide the plurality of water samples used for training. In preferred embodiments, the plurality of water samples may be derived from at least two independent sources so that the resulting paired training records span different water conditions and different absorbance levels.

Following collection, and in accordance with Step 205, one or more stock solutions and/or dilutions may be prepared from the source water. In some embodiments, water collected from each source may be placed in one or more containers, such as glass jars, which may serve as stock solutions. In some embodiments, each water sample may comprise a dilution of about 33%, about 66%, or about 100% of a source sample. In some embodiments, about four of the plurality of water samples may be derived from the same source and may comprise the same dilution (i.e., four of the samples may be replicated). It is to be appreciated that such dilution and replication schemes may be useful for generating paired training records spanning a progression from relatively low absorbance values to relatively high absorbance values. It is further to be understood that other dilution percentages and numbers of replications are contemplated in accordance with some embodiments of the present invention.

In accordance with Step 210, the water samples may be cultured and/or maintained under controlled conditions before and during data collection. In preferred embodiments, the water samples may be kept indoors under substantially identical environmental conditions for about two weeks. By way of example, the samples may be exposed to artificial light for about twenty-four hours per day while being maintained at a temperature of about 75 degrees Fahrenheit and a relative humidity of about 70%. It is to be appreciated that other controlled culturing conditions may be used in accordance with some embodiments of the present invention, provided that the conditions favorable to multiplication of the target algae or cyanobacteria for which the model is to be used.

In accordance with Step 220, aliquots (individual water samples subject to test) may be periodically taken for development of paired training records. In preferred embodiments, the paired training records may be acquired every two to three days over about two weeks. Each sampling event may include obtaining image data, in accordance with Step 223, obtaining a reference absorbance measurement from a reference spectrophotometer, in accordance with Step 224, and creating a paired training record, in accordance with Step 227. In some embodiments, the repeated sampling schedule may be selected such that the resulting paired training records span multiple stages of algal and/or cyanobacterial development and therefore represent a range of absorbance values useful for training the model. In one embodiment, at least about 200 paired observations may be collected. In preferred implementations, about 232 image/reading pairs may be obtained over multiple days.

During Step 223, image data corresponding to the spectrally dispersed image may be obtained using an imaging spectrometer as described herein. In preferred embodiments, the illumination source and image sensor may be selected and/or configured to produce and detect, respectively, light having at least a wavelength corresponding to the selected wavelength, e.g., a corresponding to the target algae or cyanobacteria photosynthesis wavelength. In some embodiments, this may include visible, far-red, and near-infrared wavelength content. Before storage and/or training, the image data may optionally be preprocessed, for example by cropping a region of interest corresponding to the dispersed spectral band, resizing, normalization, intensity correction, and/or wavelength calibration.

During Step 224, a reference absorbance measurement of the same water sample may be obtained from reference spectrophotometer 150 at the selected wavelength for the model. In preferred embodiments, the reference absorbance measurement is acquired substantially contemporaneously with capture of the spectrally dispersed image so that the reference value corresponds to the same sampled condition represented in the image data.

In accordance with Step 227, data corresponding to the spectrally dispersed image and the reference absorbance measurement may be associated and stored as a paired training record. In some embodiments, the paired training records may be stored in training/validation module 160 and/or another local or remote data repository. Each paired training record may therefore provide a supervised-learning example linking spectral image information to a spectrophotometer-derived absorbance label. In some embodiments, the machine-learning regression model may be trained “from scratch” using the plurality of paired training records rather than relying on pre-trained classification weights.

Step 230 may include building and developing machine-learning regression model 144. In preferred embodiments, machine-learning regression model 144 may be a convolutional neural network regression model configured as a sequential model. For example, and without limitation, the convolutional neural network regression model may comprise three convolutional 2D layers, two dense layers, and a dropout layer. In some embodiments, the model may be implemented in a Python-based machine-learning environment. By way of non-limiting example, the model may be programmed using Python, TensorFlow, and Keras. Although a sequential convolutional neural network regression model is preferred, other regression architectures capable of mapping spectrally dispersed image data to reference absorbance values are contemplated in accordance with embodiments of the present invention.

Step 230 may further include training the machine-learning regression model, in accordance with Step 233, and validating the machine-learning regression model, in accordance with Step 235. In preferred embodiments, training machine-learning regression model 144 may be developed to include using Adam as an optimizer, ReLU as an activation function, mean squared error as a loss function, and/or early stopping to prevent overfitting. In some embodiments, about eighty percent of the paired training records may be used as a training data set and about twenty percent of the paired training records may be used as a validation data set. During training and validation, one or more performance metrics may be monitored, such as training loss, validation loss, mean squared error, mean absolute error, and other training and validation values reported by the training environment.

During Step 235, the model may be validated by calculating predicted absorbance values for the corresponding validation samples, and comparing the predicted absorbance values with the actual absorbances measured by reference spectrophotometer 150. In some embodiments, training and validation loss curves may be reviewed to assess whether the model is learning the relationship between the image data and the reference absorbance values without unacceptable overfitting. For example, decreasing training and validation loss curves with an acceptable gap therebetween may indicate that the model has achieved effective learning. Once the desired performance has been achieved, trained machine-learning regression model 144 may be stored in memory 142 and may be incorporated into a prediction output program for later use in analyzing a new water sample not included in the training or validation data.

### Systems for Analyzing a Water Sample

According to some embodiments of the present invention, and with reference to FIGS. 1 and 2, system 100 may be configured to analyze a water sample 120 after trained machine-learning regression model 144 has been developed and validated, such as by methods for training a model disclosed herein. More particularly, system 100 may comprise illumination source 110 arranged to direct light through water sample 120, imaging spectrometer 130, and at least one processor 140 operatively engaged with memory 142. Imaging spectrometer 130 may comprise optical dispersive element 132 and image sensor 134 arranged such that the light transmitted through water sample 120 forms a spectrally dispersed image on image sensor 134. In some embodiments, imaging spectrometer 130 may further comprise image processor 136 configured to convert output from image sensor 134 into image data corresponding to the spectrally dispersed image. Processor 140 may be disposed within imaging spectrometer 130, operatively coupled to imaging spectrometer 130, or implemented in a separate computing device that communicates with imaging spectrometer 130 over a wired and/or wireless connection. Memory 142 may store instructions and trained machine-learning regression model 144 and, when the instructions are executed by processor 140, processor 140 may be caused to receive image data corresponding to the spectrally dispersed image, execute trained machine-learning regression model 144 using at least a portion of the image data as input, and output predicted absorbance value 146 for water sample 120 at a selected wavelength.

In some embodiments, the image data received by processor 140 may be generated by image processor 136 and may correspond to all or a selected portion of the spectrally dispersed image captured by image sensor 134. In some embodiments, the image data may comprise a cropped region of interest corresponding to the dispersed spectral band, and may optionally be resized, normalized, intensity-corrected, and/or wavelength-calibrated before being supplied to trained machine-learning regression model 144. In preferred embodiments, trained machine-learning regression model 144 comprises a machine-learning regression model that outputs a quantitative absorbance value rather than a classification label. In some implementations, trained machine-learning regression model 144 may comprise a convolutional neural network regression model configured as a sequential model, although it is to be appreciated that other regression architectures capable of mapping the received image data to a predicted absorbance value may be used in accordance with some embodiments of the present invention. The trained model stored in memory 142 may be the model generated using paired training records as described with respect to methods for training machine-learning models, discussed herein, including without limitation by using paired records associating spectrally dispersed image data with reference absorbance measurements obtained from optional reference spectrophotometer 150.

In operation, illumination source 110 may direct light through water sample 120 and toward imaging spectrometer 130 so that optical dispersive element 132 separates the transmitted light by wavelength and forms the spectrally dispersed image on image sensor 134. The resulting image data may then be received by processor 140 and supplied to trained machine-learning regression model 144. In response, trained machine-learning regression model 144 may output predicted absorbance value 146 at the selected wavelength for water sample 120. In some embodiments, predicted absorbance value 146 may be displayed to a user, stored in a local or remote data repository, transmitted to another device, compared with one or more thresholds, and/or provided to a prediction output program or downstream analysis routine 170. It is to be appreciated that reference spectrophotometer 150 may be used during model development and validation, but system 100 may analyze a new water sample during runtime without obtaining a contemporaneous absorbance reading from reference spectrophotometer 150.

In some implementations, illumination source 110 may comprise a full-spectrum light-emitting diode. In preferred embodiments, illumination source 110 may be dimmable so that lighting conditions may be controlled during capture of the spectrally dispersed image. In some embodiments, optical dispersive element 132 may comprise a diffraction spectroscope.

In some implementations, image sensor 134 may be adapted to detect light having wavelengths between about 300 nm and about 1,100 nm, and may comprise a camera having an infrared-cut filter removed or omitted so that imaging spectrometer 130 may capture visible light together with far-red and near-infrared wavelength content. In preferred implementations, the selected wavelength for which predicted absorbance value 146 is output may be about 680 nm, which may correspond to chlorophyll absorption. Predicted absorbance value 146 output by system 100 may be useful as an indicator associated with algae and/or cyanobacteria in water sample 120, and when collected over time, may be used as a proxy for changes in algal or cyanobacterial activity and may support evaluation of bloom-related conditions, early warning, and/or harmful algal bloom monitoring.

It is to be understood that variations, modifications, and permutations of embodiments of the present invention may be made without departing from the scope thereof. It is also to be understood that the present invention is not limited by the specific embodiments, descriptions, or illustrations or combinations of either components or steps disclosed herein. Thus, although reference has been made to the accompanying figures, it is to be appreciated that these figures are exemplary and are not meant to limit the scope of the invention.

Moreover in this document, relational terms, such as second, top and bottom, and the like may be used solely to distinguish one entity or action from another entity or action without necessarily requiring or implying any actual such relationship or order between such entities or actions. The terms “comprises”, “comprising”, “has”, “having,” “includes”, “including”, “contains”, “containing”, or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises a”, “has . . . a”, “includes . . . a”, “contains . . . a” does not, without more constraints, preclude the existence of additional elements of the same type in the process, method, article, or apparatus that comprises, has, includes, contains the element. The terms “a” and “an” are defined as one or more unless explicitly stated otherwise herein. The terms “substantially”, “essentially”, “approximately”, “about”, or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” or “connected” as used herein may not necessarily indicate a direct connection or not necessarily mechanical connection. Methods or processes disclosed herein may be embodied in hardware and/or software. Similarly, the term “processor” may refer to hardware and/or software. A device or structure that is “configured” in a certain way is configured in at least that way, but may also be configured in ways that are not listed. Also, the term “exemplary” is used as an adjective herein to modify one or more nouns, such as embodiment, system, method, device, and is meant to indicate specifically that the noun is provided as a non-limiting example.

What is claimed is:

1. A method of analyzing a water sample, said method comprising:
a capturing, with an imaging spectrometer comprising an image sensor and an optical dispersive element, a spectrally dispersed image of light transmitted through said water sample, wherein said optical dispersive element is arranged such that said light transmitted through said water sample forms said spectrally dispersed image on said image sensor;
b) generating image data corresponding to said spectrally dispersed image;
c) providing at least a portion of said image data to a trained machine-learning regression model; and
d) outputting, with said trained machine-learning regression model, a predicted absorbance value of said water sample at a selected wavelength.

2. The method of claim 1, wherein said step of capturing said spectrally dispersed image comprises illuminating said water sample with a full-spectrum light-emitting diode.

3. The method of claim 1, wherein said optical dispersive element comprises a diffraction spectroscope.

4. The method of claim 1, wherein said image sensor is adapted to detect light having wavelengths between about 300 and about 1,100 nm.

5. The method of claim 1, wherein said image data includes wavelength content in a range from about 300 nm to about 1,100 nm.

6. The method of claim 1, wherein said selected wavelength is about 680 nm.

7. The method of claim 1, wherein said selected wavelength corresponds to chlorophyll absorption.

8. The method of claim 7, further comprising using said predicted absorbance value to predict cyanobacteria concentration in said water sample.

9. A method of training a machine-learning model to predict absorbance of a water sample at a selected wavelength, said method comprising:
a for each of a plurality of water samples,
1) capturing, from an imaging spectrometer comprising an image sensor and an optical dispersive element, a spectrally dispersed image of light transmitted through said water sample;
2) obtaining, from a reference spectrophotometer, a reference absorbance measurement of said water sample at said selected wavelength;
3) storing a paired training record comprising data corresponding to said spectrally dispersed image and said reference absorbance measurement; and
b) training, using said paired training records, a machine-learning regression model configured to output a predicted absorbance value at said selected wavelength.

10. The method of claim 9, wherein said selected wavelength is about 680 nm.

11. The method of claim 9, wherein said paired training records are acquired every two to three days over about two weeks.

12. The method of claim 9, wherein each said water sample is derived from one of at least two independent sources.

13. The method of claim 12, wherein each said water sample comprises a dilution of one of about 33%, about 66%, and about 100% of said source.

14. The method of claim 13, wherein about four of said plurality of water samples are derived from the same source and comprise the same dilution.

15. The method of claim 9, wherein said machine-learning regression model comprises a convolutional neural network regression model configured as a sequential model.

16. The method of claim 15, wherein said convolutional neural network regression model comprises three convolutional 2D layers, two dense layers, and a dropout layer.

17. The method of claim 16, wherein training said machine-learning regression model comprises using Adam as an optimizer, ReLU as an activation function, mean squared error as a loss function, and early stopping to prevent overfitting.

18. The method of claim 17, wherein about eighty percent (80%) of said paired training records are used as a training data set and about twenty percent (20%) of said paired training records are used as a validation data set.

19. A system for analyzing a water sample, said system comprising:
a) an illumination source arranged to direct light through said water sample;
b) an imaging spectrometer comprising an image sensor and an optical dispersive element arranged such that said light transmitted through said water sample forms a spectrally dispersed image on said image sensor; and
c) at least one processor operatively engaged with a memory storing instructions that, when executed by said at least one processor, cause said at least one processor to:
1) receive image data corresponding to said spectrally dispersed image,
2) execute a trained machine-learning regression model using at least a portion of said image data as input, and
3) output a predicted absorbance value for said water sample at a selected wavelength.

20. The system of claim 19, wherein said illumination source comprises a full-spectrum light-emitting diode, wherein said optical dispersive element comprises a diffraction spectroscope, wherein said image sensor is adapted to detect light having wavelengths between about 300 and about 1,100 nm, and wherein said selected wavelength is about 680 nm.
